(19) United States
(12) Patent Application Publication
(10) Pub. No.: US 2025/0333720 A1
(43) Pub. Date: Oct. 30, 2025

(54) CEREBRAL ORGANOID AND ELECTRICAL STIMULATION PREPARATION METHOD THEREOF

(71) Applicant: SHENZHEN INSTITUTES OF ADVANCED TECHNOLOGY CHINESE ACADEMY OF SCIENCES, Shenzhen (CN)

(72) Inventors: Qian LIU, Shenzhen (CN); Yan WANG, Shenzhen (CN); Bing SONG, Shenzhen (CN)

(73) Assignee: SHENZHEN INSTITUTES OF ADVANCED TECHNOLOGY CHINESE ACADEMY OF SCIENCES, Shenzhen (CN)

(21) Appl. No.: 19/260,437

(22) Filed: Jul. 4, 2025

Related U.S. Application Data
(63) Continuation of application No. PCT/CN2023/095227, filed on May 19, 2023.

(51) Int. Cl. C12N 13/00; C12N 5/0793

(52) U.S. Cl. CPC C12N 13/00; C12N 5/0619; C12N 2506/45; C12N 2529/00

(57) ABSTRACT

The invention relates to the technical field of cell culture, and provides an electrical stimulation preparation method for cerebral organoids, wherein the electrical stimulation preparation method for cerebral organoid comprises the following steps: S1: adopting a conventional cerebral organoid culture medium to culture stem cells, and applying sinusoidal alternating current stimulation in the culture process; S2: continuing to culture the stem cells until the cerebral organoid with a basic structure is formed after the electrical stimulation is completed.

### TECHNICAL FIELD

The invention belongs to the technical field of cell culture, and particularly relates to a cerebral organoid and an electrical stimulation preparation method thereof.

### BACKGROUND

Since the development of stem cells, especially induced pluripotent stem cells (iPSCs) in the 1980s, stem cell technology has been widely used in the study of physiological mechanisms and pathological mechanisms of diseases, tissue and organ transplantation and regeneration, and clinical applications. The central nervous system composed of brain and spinal cord is an important functional tissue and organ of human body. Due to the particularity of its nervous function, blood-brain barrier and anatomical structure, the clinical diagnosis, mechanism exploration and treatment research of brain diseases are particularly difficult. With the rapid development of stem cell technology, neural cells derived from embryonic stem cells (ESCs), neural stem cells (NSCs) and iPSCs can be used to study the pathological and therapeutic mechanisms of brain physiology and disease models.

Stem cells were initially used in the study of brain diseases, starting from the induction of differentiation of ESCs, NSCs and iPSCs cultured in two-dimensional culture dishes into the main functional cells in the brain: neurons, astrocytes, oligodendrocytes and so on. The main mechanism of this inducible regulation is to guide the proliferation and differentiation of stem cells through gene editing, growth factors and signal factors. However, such two-dimensional induced cultivation mechanism lacks the corresponding multi-cell type internal environment and cell-cell interaction in brain tissues and organs, which are essential for the simulation of brain nerve function. In order to solve this problem, the brain application research of stem cells has turned to another way of differentiation: to establish the induction mode of stem cells in 3D culture, and to form the regional development of various nerve cell types, that is, to prepare cerebral organoids.

Cellular architecture and interactions of cellular network in its 3D environment are critical for brain function. Therefore, in order to better simulate brain tissue in a culture dish, it is crucial to develop 3D stem cell-derived cerebral organoids. Studies have shown that mouse iPSCs cultured in serum-free suspension formed 3D tissues of neuroectodermal origin. This neuroectodermal-like epithelial cell differentiates into multiple layers of cortical tissue, containing progenitor cells and neurons. These may be considered the first true central nervous organoids: 3D retinal structures with layered neuroepithelial cells containing all major retinal cell types. They subsequently demonstrated the differentiation of human iPSCs cultures to the retina and various neural 3D structures. Differentiation of the forebrain, neocortex, and hippocampal lineages was achieved by the temporary addition of agonists or inhibitors of growth factors, FGF, Wnt, and BMP signal networks. Thus, studies have shown that organoid cultures derived from iPSCs are able to respond to biochemical and biomechanical stimuli and, through diversification of these cues, derive organotypic cultures of different central nervous structures, including the brain, spinal cord, and retina.

The existing preparation methods of cerebral organoids are mainly regulated by cytokines and signaling pathway factors. Manipulating intracellular signals such as Sonic Hedgehog patterns the forebrain organoids into dorsal and ventral subdomains that better mimic the in vivo topology. Treatment with brain-derived neurotrophic factor (BDNF), glial cell line-derived neurotrophic factor (GDNF), and ascorbic acid can guide differentiation towards the midbrain and brainstem lineages (Eura et al., 2020). Continuous BMP and SMAD inhibition and activation promote continuous differentiation and maintenance of dopaminergic neurons in midbrain organoids. Alternatively, addition of WNT3 A and the Hedgehog pathway agonist Purmorphamine can induce the hypothalamic lineage at the neuroectodermal stage. After 4 weeks in vitro, the brain organoids showed regional features such as FOXG1 delineating the forebrain, while TTR expression delineated the choroid plexus and FZD9 hippocampus-like regions. However, the existing preparation methods of cerebral organoids often have shortcomings in the actual preparation. For example, whether the cerebral organoids are prepared by induction or non-induction methods, their maturity as central nervous systems is limited, the culture time consumed is long, and the reliance on autonomous differentiation will lead to batch-to-batch variation in differentiation and heterogeneity of cerebral organoids; cerebral organoid preparation methods that rely on the self-organization of iPSCs and the stochastic nature of their spontaneous differentiation lead to inherently variable results. Therefore, it is different from organs produced through a precisely controlled process of organogenesis in the body.

### SUMMARY

In order to improve the success rate of neurogenesis, accelerate the preparation rate of cerebral organoids, reduce the differentiation heterogeneity and realize the precise control in the organogenesis of cerebral organoids, the invention provides an electrical stimulation preparation method for preparing cerebral organoids.

The electrical stimulation preparation method for cerebral organoid is characterized by comprising the following steps:
S1: adopting a conventional cerebral organoid culture medium to culture stem cells, and applying sinusoidal alternating current electrical stimulation in the culture process.
S2: continuing to culture the stem cells until the cerebral organoid with a basic structure is formed after the electrical stimulation is completed.

Further, the electrical stimulation is sinusoidal alternating current electrical stimulation with the frequency of 0-130 Hz.

Further, the voltage strength is 200 mV/mm.

Further, the electrical stimulation time is 1 hour/day;

The stimulation is continued for 14 days.

Further, the frequency of the sinusoidal alternating current electrical stimulation is 130 Hz;

The voltage strength is 200 mV/mm;

The electrical stimulation time is 1 hour/day;

The cultivation period lasts for 14 days.

Further, the stem cell is selected from one of human-derived iPSCs, human-derived ESCs and mouse-derived NSCs.

One objective of the present invention is to provide a type of cerebral organoid. The cerebral organoid is prepared by the electrical stimulation preparation method for the cerebral organoid according to any one of the above descriptions.

An object of the present invention is to provide applications of cerebral organoids in drug screening, disease research, tissue and organ transplantation and regeneration research, or cell research.

With the electrical stimulation preparation method for cerebral organoid, the cerebral organoid maturation time is shortened to 28-40 days; and the proportion of neuron cells in the prepared cerebral organoid tissue is increased from 8% to 14%, the success rate of neurogenesis is improved, the preparation speed of the cerebral organoid is accelerated, the differentiation heterogeneity is reduced, and the cerebral organoid tissue has high similarity with the organ generated in the precise control process of in vivo organogenesis.

### BRIEF DESCRIPTION OF DRAWINGS

FIG. 1 illustrates a device for preparing cerebral organoid according to the present invention;

FIG. 2 illustrates a flow diagram of a method for preparing cerebral organoid according to the present invention;

FIG. 3 illustrates a schematic diagram of a method for preparing cerebral organoid according to the present invention;

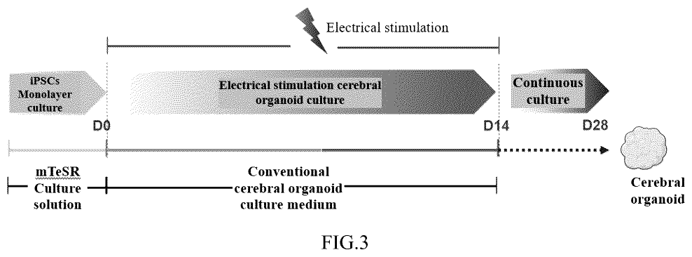

FIG. 4 illustrates a test result of the prepared cerebral organoid prepared in Embodiment 1.

### DETAILED DESCRIPTION OF THE EMBODIMENTS

In order that the above objects, features and advantages of the present invention may be more clearly understood, the specific implementation modes of the present invention are described in detail below with reference to the accompanying drawings, but they should not be understood as limiting the scope of the present invention.

Studies on embryonic development have confirmed that physiological electric fields play an important role in embryonic neurogenesis. In the process of neural tube formation, the spatial difference formed by the bulge of neural ridge produces the voltage difference across the neural tube, that is, the physiological electric field. The early amphibian neural tube has a direct current electric field as high as 450-1600 mV/mm, which is measured by glass electrodes and non-invasive oscillating microelectrodes, and neuroblasts are differentiated in this physiological electric field generated by the neural tube. Studies have shown that experimental blocking of physiological electric fields can lead to abnormal development of the central nervous system, such as closure and failure of the neural tube, and lack of central nervous system structure. The research group in Japan used physiological electric field (125-500 mV/mm) to stimulate ESCs to differentiate into embryoid bodies in vitro, and analyzed the phenotype of differentiated cells in the embryoid bodies. The results showed that the physiological electric field applied at the embryoid body stage could significantly promote the differentiation of embryonic stem cells into neurons; Studies also showed that physiological intensity of direct current electrical stimulation (100-150 mV/mm) could effectively regulate the differentiation of NSCs into neurons in vitro; and the neurite branching and length induced by electrical stimulation were similarly found in the study of exogenous electrical stimulation applied to NPC derived from fetal mice and immortalized human brain. Sufficient neurite branching is important for the formation of functional neuronal networks, suggesting that electrical stimulation may also enhance the structural and functional establishment of in vitro neurodevelopmental models. In summary, these findings suggest that electrical stimulation regulation may promote cell cycle related effects (proliferation, survival, and differentiation) in nerve cells to help reproduce neurogenesis from stem cells. In view of the consistent reports that electrical stimulation can induce and enhance neurogenesis, improve cell survival and reduce cell death, it provides hope for cerebral organoid culture. Therefore, the application of electrical stimulation during cerebral organoid preparation may be an effective method to improve the success rate of neurogenesis, accelerate the rate of cerebral organoid preparation and reduce the differentiation heterogeneity. In addition, iPSCs that are electrically stimulated at the early stages of brain organoid differentiation may provide more robust neuroectoderm and subsequent induction of neural lineage cells, so applying electrical stimulation during cerebral organoid preparation may be an effective way to improve the success rate of neurogenesis, accelerate the rate of cerebral organoid preparation and reduce differentiation heterogeneity.

In summary, electrical stimulation can enhance neural network and tissue development in vitro and in vivo. Although there is substantial evidence that endogenous electrical stimulation promotes neurogenesis, researchers have not explored the potential of bioelectrical signals in directing iPSCs-derived cerebral organoids. Proof-of-principle studies on mouse and human iPSCs and ESCs support the ability to use electrical stimulation for neural culture at the iPSCs stage without impeding differentiation capacity. The biological processes regulated by electrical stimulation in NSCs cultures are closely related to the processes involved in generating and optimizing iPSCs derived organoids.

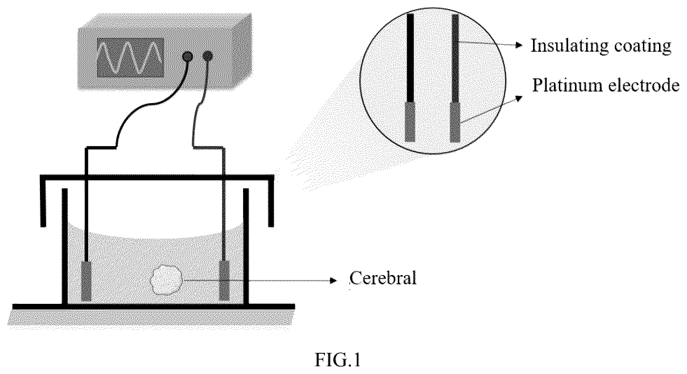

Referring to FIG. 1 of the specification, the present invention provides a device for preparing cerebral organoid for culturing cerebral organoid. The device comprises an electrical stimulation culture dish, a cover plate, a power supply and a stimulation electrode. The electrical stimulation culture dish is used for placing stem cells and a culture medium; the cover plate is covered on the electrical stimulation culture dish to form a closed space, and the cover plate is provided with a through hole; the electrical stimulation electrode penetrates through the through hole and enters the closed space; and the power supply is electrically connected to the stimulation electrode.

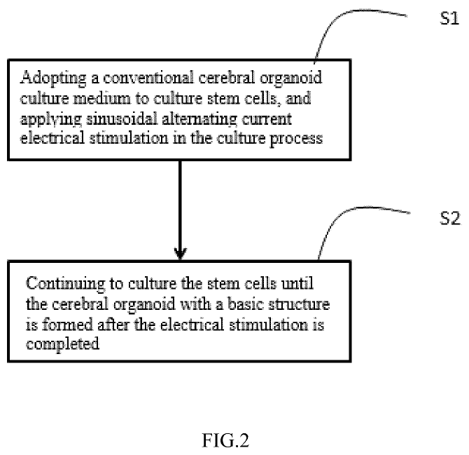

Referring to FIGS. 2&3 of the Specification, the present invention provides an electrical stimulation preparation method for cerebral organoid, comprising the following steps:

S1: Adopting a conventional cerebral organoid culture medium to culture stem cells, and applying sinusoidal alternating current electrical stimulation in the culture process.

A conventional cerebral organoid culture medium is a commercial induction culture medium, and customized (STEMdiff™ Cerebral Organoid Kit) cytokines and additives are added. The stem cell is selected from one of human-derived iPSCs, human-derived ESCs and mouse-derived NSCs. The stem cells used in the present application were obtained from stem cells cultured in mTeSP medium.

S2: continuing to culture the stem cells until the cerebral organoid with a basic structure is formed after the electrical stimulation is completed.

After the electrical stimulation, the stem cells are continuously cultured in a conventional cerebral organoid culture medium for differentiation and maturation to form cerebral organoid.

Stem cells that are electrically stimulated by sinusoidal alternating current in the early stages of brain organoid differentiation are induced to produce more robust neuroectoderm and subsequent neural lineage cells, so electrical stimulation during organoid preparation is an effective way to improve the success rate of neurogenesis, accelerate the rate of cerebral organoid preparation and reduce differentiation heterogeneity. Therefore, the generated cerebral organoids have higher similarity with the organs generated in the precise control process of organogenesis in vivo.

In one embodiment, the electrical stimulation preparation method for cerebral organoid comprises the following steps:

S1: culturing stem cells by adopting a conventional cerebral organoid culture medium, and applying sinusoidal alternating current electrical stimulation during the culture process, wherein the frequency is 10-130 Hz, preferably 10-130 Hz, the voltage strength is 200 mV/mm, and the electrical stimulation time is 1 hour/day; and the stimulation is continued for 14 days.

The induction scheme relies only on the self-organization and spontaneous differentiation of stem cells to produce the randomness of the structure and organization of cerebral organoids. The application aims to apply electrical stimulation in the process of stem cell induction and culture to generate an organ with a specific structure and tissue, and improve the similarity of the organ generated under the precise control in vivo, so that the generated cerebral organoid can be used for the research and clinical application of various physiological mechanisms, pathological mechanisms of diseases, tissue and organ transplantation and regeneration. The tissue and structure of the cerebral organoid to be prepared is similar to that of an organ produced under precise control in vivo.

In this application, the electrical stimulation time is limited to 1 hour/day for 14 consecutive days. On the one hand, it can improve the success rate of neurogenesis, accelerate the preparation rate of cerebral organoids, and reduce differentiation heterogeneity. On the other hand, it can ensure that stem cells have a high survival rate.

S2: continuing to culture the stem cells for 14-26 days until the cerebral organoid with a basic structure is formed after the electrical stimulation is completed.

The time of continuous culture can be adjusted according to the maturity of the cerebral organoid.

### Embodiment 1

An electrical stimulation preparation method for cerebral organoid comprises the following steps:
S1: culturing human-derived induced pluripotent stem cells (iPSCs) by adopting a conventional cerebral organoid culture medium, and applying sinusoidal alternating current electrical stimulation from the 1st day to the 14th day of induced culture, wherein the frequency is 130 HZ, and the voltage strength is 200 mV/mm; the time is 1 hour/day, and the continuous stimulation is performed for 14 days.
S2: continuing to culture for 28-40 days after the electrical stimulation is finished to form the cerebral organoid with the diameter of more than 300 μm and a basic structure.

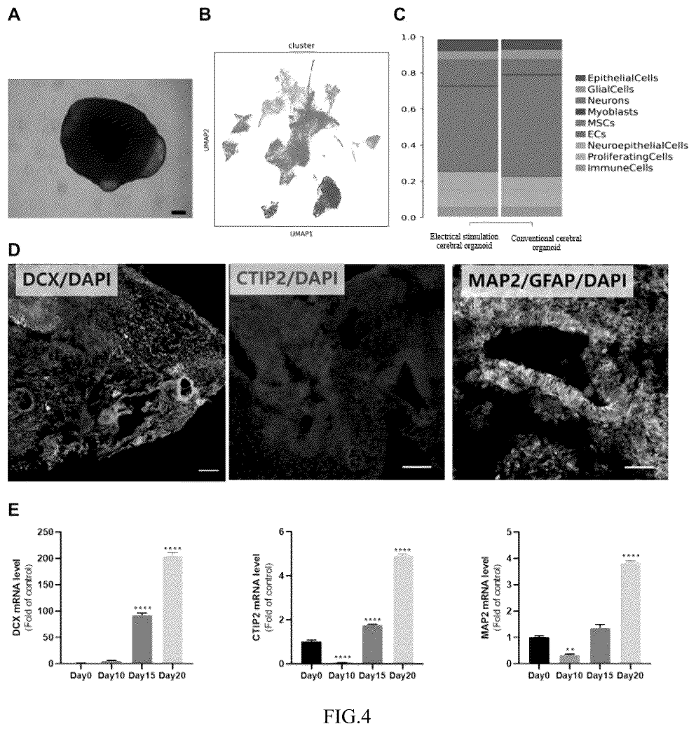

The success of cerebral organoid preparation was identified by single cell sequencing, marker gene and protein expression detection, and the results are shown in FIG. 4. In FIG. 4, A represents a microscope photograph of the cerebral organoid prepared by electrical stimulation; B represents a comparison of the single cell sequencing of the cerebral organoid prepared by electrical stimulation and conventional culture; C shows that the proportion of neurons in the cerebral organoid prepared by electrical stimulation is 14%, while that in conventional culture is only 8%; D represents the immunofluorescence identification of the cerebral organoid cultured by electrical stimulation, wherein DCX, CTIP2 and MAP2 are the marker genes of neuron cells, and GFAP is the marker gene of glial cells; and E represents the real-time quantitative PCR identification of the cerebral organoid culture by electrical stimulation, and the result shows that the expression of the neuron marker gene of the cerebral organoid culture on the 15th day of the induction culture is significantly enhanced after 14 days of the electrical stimulation culture.

The cerebral organoid induction culture maturation time of a classical induction scheme and a non-induction scheme needs 40-70 days, but the invention adopts an electrical stimulation preparation method for a self-made electrode and a culture dish plate, so that the cerebral organoid maturation time is shortened to 28-40 days; and meanwhile, the proportion of neuron cells in the prepared cerebral organoid tissue is increased from 8% to 14%.

What is claimed is:

1. An electrical stimulation preparation method for cerebral organoid, characterized by comprising the following steps:
S1: adopting a conventional cerebral organoid culture medium to culture stem cells, and applying sinusoidal alternating current electrical stimulation in the culture process;
S2: continuing to culture the stem cells until the cerebral organoid with a basic structure is formed after the electrical stimulation is completed.

2. The electrical stimulation preparation method for cerebral organoid according to claim 1, wherein the electrical stimulation is sinusoidal alternating current electrical stimulation with the frequency of 0-130 Hz.

3. The electrical stimulation preparation method for cerebral organoid according to claim 1,
wherein the voltage strength is 200 mV/mm.

4. The electrical stimulation preparation method for cerebral organoid according to claim 1,
wherein the electrical stimulation time is 1 hour/day; and
the stimulation is continued for 14 days.

5. The electrical stimulation preparation method for cerebral organoid according to claim 1,
wherein the frequency of the sinusoidal alternating current electrical stimulation is 130 Hz;
the voltage strength is 200 mV/mm;
the electrical stimulation time is 1 hour/day; and
the cultivation period lasts for 14 days.

6. The electrical stimulation preparation method for cerebral organoid according to claim 1, wherein the stem cell is selected from one of human-derived iPSCs, human-derived ESCs and mouse-derived NSCs.

7. A cerebral organoid, prepared by the electrical stimulation preparation method according to claim 1.

8. Application of the cerebral organoid according to claim 7 in drug screening, disease research, tissue and organ transplantation and regeneration research, or cell research.
